(19) United States
(12) Patent Application Publication
(10) Pub. No.: US 2018/0078411 A1
(43) Pub. Date: Mar. 22, 2018

(54) OPHTHALMOLOGICAL LASER THERAPY DEVICE AND METHOD FOR CALIBRATION

(71) Applicant: Carl Zeiss Meditec AG, Jena (DE)

(72) Inventors: Jochen Fuchs, Neu-Ulm (DE); Thomas Hamann, Jena (DE)

(73) Assignee: Carl Zeiss Meditec AG, Jena (DE)

(21) Appl. No.: 15/553,165

(22) PCT Filed: Feb. 23, 2016
(86) PCT No.: PCT/EP2016/053709

§ 371 Date: Aug. 23, 2017

(30) Foreign Application Priority Data
Feb. 27, 2015 (DE) 102015002726.3

(51) Int. Cl. A61F 9/008; A61B 3/10

(52) U.S. Cl. CPC A61F 9/008; A61B 3/102; A61N 5/06; A61F 2009/00855; A61F 2009/00851

(57) ABSTRACT

An ophthalmological laser treatment device including a laser system, an examination system for collecting information on the structure of the eye, a positioning system for controlling a therapy laser beam and electromagnetic or mechanical examination waves and a patient interface. The invention further relates to a calibration method and to a treatment method. The invention provides as device and a method, by application of which calibration of treatment laser beam and electromagnetic or mechanical examination waves and optionally optical images can be carried out automatably and repeatedly. An ophthalmological laser treatment device, includes a detection system having a detector and an observation volume configured for the repeatable spatially resolving detection and joint representation of the signals of the treatment laser beam striking the observation volume and the electromagnetic or mechanical examination waves, by corresponding calibration method and laser treatment method, and by a patient interface.

### RELATED APPLICATIONS

This application is a National Phase entry of PCT Application No. PCT/EP2016/053709 filed Feb. 23, 2016 which application claims the benefit of priority to German Application No. 10 2015 002 726.3, filed Feb. 27, 2015, the entire disclosures of which are incorporated herein by reference.

### FIELD OF THE INVENTION

The present invention relates to an ophthalmological laser therapy device comprising a laser system for treating tissue of an eye, configured to generate a therapy laser beam in a first frequency, an examination system for compiling data on the structure of the eye by application of electromagnetic or mechanical examination waves having a second frequency, and a positioning system, configured to control the therapy laser beam and the electromagnetic or mechanical examination waves. The invention also relates to a patient interface for an ophthalmological therapy device, a calibration method for an ophthalmological laser therapy device, and an ophthalmological therapy method.

### BACKGROUND

With the introduction of laser systems in eye surgery, for which cataract surgery represents, by way of example, one of the most frequently used eye surgery methods, the classic scalpel is replaced in this procedure by a laser. High demands are placed on precision thereby. As before, visual recordings of the eye are used for planning and executing the operation as precisely as possible. For a precise operation, the focal position of the treating laser beam must be determined in relation to electromagnetic examination waves of one or more examination systems, e.g. an optical coherence tomography (OCT) system or an ultrasound system, or alternatively, 3-D recordings with other methods and/or, if applicable, in relation to different visual recordings. It is thus essential to establish a clear correlation between the systems. This makes it possible to use the results of the examination of the state of the eye prior to the operation with such an examination system and/or with visual recordings, in order to create a treatment plan for the ophthalmological laser therapy, in order to actually execute the laser incisions with the laser therapy beam during the operation at the location defined, by way of example, in the OCT and visual observation recordings, and to track the course of the treatment.

This applies in particular when different beam paths are used for the visual recordings and the OCT imaging.

Normally, visual recordings of the eye, in particular the iris and the lens, are made first, using a camera. On the basis of these recordings, the operator can determine regions in which further recordings need to be made using optical coherence tomography or other imaging methods. The laser incisions are planned subsequently on the basis of this data acquired prior to the laser therapy.

Known solutions require a one-time adjustment of visual observations or visual recordings, an OCT laser beam and therapy laser beam, which then must be maintained. In the known solutions, starting at the point in time at which the patient, or the eye of the patient, is brought into position, it is not possible to control the position of the therapy laser beam prior to or during the operation. As a result, the risk to the patient, of a laser incision taking place at an unintended location, increases. If it becomes misadjusted, such ophthalmological laser therapy systems must be readjusted manually, and the operation itself must be aborted.

In US 2009/0131921 A1, which describes a laser therapy system for cataract surgery, the laser therapy beam, visual recordings in the visible spectrum, and the OCT recordings are coordinated to one another, in that an external calibration sample is introduced and used in the system prior to the surgical operation, and into which reference marks are etched with the therapy laser beam. During the subsequent surgical operation, this calibration cannot be verified, because the calibration sample can no longer be used.

In particular when the course of the operation is actively observed during the use of the laser therapy beam, and intervention is required in the course of the treatment on the basis of these observations, a determination, however, of the relative geometric position of the focal position by electromagnetic or mechanical examination waves of one or more examination systems, as well as the signals of visual recordings in the visible spectrum regarding the therapy laser beam, the knowledge of the relative movements of the systems in relation to one another, and the possibility of monitoring the calibration and a periodic repetition of the calibration process, also during an operation, is of fundamental importance. The fact that the therapy laser beam, the visual recordings, OCT recordings, or alternatively, three dimensional recordings, function with light, or electromagnetic or mechanical examination waves of different wavelengths, and thus different frequencies, significantly complicates such a calibration, and active control or correction of the calibration during the course of the operation.

### SUMMARY

Embodiments of the present invention create an ophthalmological laser therapy device and a calibration method for the ophthalmological laser therapy device, as well as an ophthalmological laser therapy method, with which the calibration of therapy laser beams and electromagnetic or mechanical examination waves of examination systems and, if applicable, visual recordings in the visible spectrum can be repeated and executed automatically, without having to interrupt and restart the therapy procedure.

An ophthalmological laser therapy device comprises a laser system, which is configured to generate a therapy laser beam, thus from electromagnetic waves in a first frequency, wherein the therapy laser beam is used to treat eye tissue.

The ophthalmological laser therapy device furthermore comprises at least one examination system, which is configured to compile data regarding the structure of the eye by application of electromagnetic or mechanical examination waves in a second frequency. The first and/or second frequencies, and/or any other frequencies described herein refer to a mean frequency; depending on the type of electromagnetic or mechanical examination wave, these waves may contain a more or less broad frequency spectrum surrounding this first, second or further frequency. The electromagnetic or mechanical examination waves are first directed, for example starting from their source, at least before they strike structures of the eye, or another conversion or diffusion object. They can for example be focused.

With the use of laser radiation in a therapy laser beam, the laser radiation normally has a very narrow frequency range (monochromatic light) in the near infrared range (NIR), or in the infrared range (IR). If, however, an OCT system is used as the examination system, it is then possible to use a very wideband laser radiation, thus laser radiation having a wide frequency range. Normally with the use of an OCT system, the laser radiation is in the near infrared range (NIR), or in the infrared range (IR).

The first frequency and the second frequency, as well as every other frequency, normally differ from one another. There are however, special systems in which the first and second frequencies as well as other frequencies among themselves—in terms of their property as a mean frequency—have the same value.

The examination system of the ophthalmological laser therapy device is for example an optical coherence tomography (OCT) system. The OCT system is configured to generate electromagnetic examination waves in the form of an examination laser beam, in particular to generate focused electromagnetic examination waves in the form of a focused examination laser beam. OCT systems belong to the most frequently used examination systems in ophthalmology, and offer the advantage of a comprehensive spatial examination of the eye in three dimensional space with greater precision. As a matter of course, instead of an OCT system, or in addition to an OCT system, an ultrasound and/or a Scheimpflug camera, for example, can also be used.

Furthermore, the ophthalmological laser therapy device comprises a positioning system, which is configured to control the therapy laser beam and the electromagnetic or mechanical examination waves. The control of the therapy laser beam and the electromagnetic or mechanical examination waves can take place thereby independently of one another, or with a shared deflection system.

According to example embodiments of the invention, the ophthalmological laser therapy device comprises a detection system, which contains a detector and an observation volume, and is configured for spatially resolved detection and the collective depiction, that can be repeated at any time, of signals of the therapy laser beam and the electromagnetic or mechanical examination waves in an observation plane of the observation volume, or in the entire observation volume, and thus also the collective depiction of their orientations and/or their focus positions in an example embodiment, as well as their relative positions to one another. A calibration is thus also possible in the course of an ophthalmological laser therapy procedure, without having to abort the therapy.

The detection system thus includes an observation volume permanently belonging to the ophthalmological laser therapy device, which is always available, at least in part, in order to determine the relative positions of the signals of the therapy laser beam and the electromagnetic or mechanical examination waves in relation to one another.

At least a portion of the components of the detection system is positioned thereby permanently in the beam path or the course of the wave. Components of the detection system that are not permanently positioned in the beam path or course of the wave can be guided repeatedly into the beam path or course of the wave.

If an observation plane of the observation volume is used for the depiction, then this corresponds advantageously to a treatment plane of the laser therapy. The observation plane can be moved thereby in the overall observation volume. For example, this movement takes place along an optical axis of the laser therapy device.

The spatially resolved detection of the signals of the therapy laser beam and the electromagnetic or mechanical examination waves may take place thereby simultaneously, or sequentially inside very short time intervals in the millisecond or microsecond range, wherein for the latter variation, this takes place in a time interval through which a desired wavelength range can pass.

Because the symbols can be depicted collectively, the data for positioning these signals in an observation plane of an observation volume, or in the overall observation volume, are available in a collective reference system.

This is a prerequisite for a simple and low error rate calibration of the therapy laser beam to the electromagnetic or mechanical examination wave examination system, thus the intentional influencing of their relative positions to one another.

As a result, a relative relationship between the therapy laser beam and the electromagnetic or mechanical examination waves of the examination system is established and can be tracked continuously, even when the positions of the therapy laser beam and the electromagnetic or mechanical examination waves are modified by the positioning system.

Correction values for a calibration can be calculated manually thereby with the determined positions of the signals. Alternatively, this step can also take place automatically, by application of a calibration system.

The constantly possible determination of the relative relationship between the therapy laser beam and the electromagnetic or mechanical examination waves is a prerequisite for a repeatable automatic calibration.

It may not be necessary thereby to actually visually depict the positions of the signals collectively. A depiction or output of just the coordinates of the signal positions of therapy laser beams and electromagnetic or mechanical examination waves in an observation plane of an observation volume or in the overall observation volume as a function of the parameters of the positioning system is also possible. However, the visual collective depiction of the signal positions of the therapy laser beam and the electromagnetic or mechanical examination waves is also a decisive aid for the surgeons or therapists, such that use is also normally made of a visual depiction of this type.

There are numerous possibilities for depicting the signals collectively: Because these are radiation or waves having different frequencies, i.e. having different wavelengths, the detection system is configured such that the radiation or waves of different frequencies from every position in the observation volume can be detected with the same detector in a spatially resolved manner, and a digital processing of the signals takes place, i.e. the detector is configured for spatially resolved spectral detection, or the radiation or waves having different frequencies are converted into radiation or waves having a common frequency, and this frequency can be detected in a spatially resolved manner by the detector. The detector thus serves as an intermediary between the otherwise potentially entirely independently acting laser system of the therapy laser beam and the examination system.

It is advantageous thereby when the ophthalmological laser therapy device includes, for example, a beam splitter or a reflector, which enables a deflection of the therapy laser beam, and/or the electromagnetic or mechanical examination waves that are to be detected, toward the detector. Without such a beam splitter or reflector, it is not possible to detect the therapy laser beam when it is directed perpendicularly, or detect a perpendicular incidence of the electromagnetic or mechanical examination waves, in the observation plane of the observation volume.

The-detection system of the ophthalmological laser therapy device may be configured such that comparison images can be determined. As a result, it is possible to generate a comparison image from the detection of a state in which the therapy laser beam and/or the electromagnetic or mechanical examination waves are switched off, and the detection of a state in which the therapy laser beam and/or the electromagnetic or mechanical examination waves are switched on, illustrating the differences of the images of the two states, in order to determine the positions of the signals from the laser therapy beam and electromagnetic or mechanical examination waves, in particular in order to also be able to define the focal point of the beam and its divergence or the course of the wave. The comparison image does not need to be applied physically thereby, but rather, the knowledge of the differences in values for each point of the observation volume or an observation plane in an observation volume is sufficient.

Alternatively, a determination of the positions of the signals from the therapy laser beam and electromagnetic or mechanical examination waves is also possible using an algorithmic comparison, thus using templates and an image recognition that supports templates.

In an example design, the detection system of the ophthalmological laser therapy device includes a camera system, which is configured to generate a visual recording of structures of the eye by application of electromagnetic waves having a third frequency from the visible spectrum, and for spatially resolved detection and collective depiction of signals of electromagnetic waves of the third frequency striking an observation plane of the observation volume or in the overall observation volume, and signals of the therapy laser beam and/or the electromagnetic or mechanical examination waves of the second frequency.

As a result, it is possible to depict the positions of signals of the therapy laser beam and the electromagnetic or mechanical examination waves in the visual recordings of the camera system. The therapist or an automatic control system can thus map these signals directly, and intervene immediately when a deviation from the expected course is observed.

In another design, the ophthalmological laser therapy device includes a detection system configured to detect signals of different frequencies of electromagnetic examination waves ranging from a frequency range of microwaves or infrared light to the entire range of visible light. Alternatively or at the same time, the detection system can also detect mechanical examination waves in the ultrasound frequency range.

For the electromagnetic examination waves, the frequency range of approx. 10⁸ Hz to approx. 10¹² Hz comprises microwaves, the frequency range of approx. 10¹² Hz to approx. 3.75*10¹⁴ comprises infrared light, of which the frequency range of approx. 3*10¹³ Hz to approx. 3.75*10¹⁴ Hz comprises the near infrared light, and the frequency range of approx. 3.75*10¹⁴ Hz to approx. 7.9*10¹⁴ Hz comprises visible light, the frequency range of approx. 7.9*10¹⁴ Hz to approx. 10¹⁷ Hz comprises ultraviolet light, and the frequency range of approx. 10¹⁷ Hz to approx. 10²¹ Hz comprises X-rays. For the mechanical examination waves, the frequency range of approx. 16 kHz to approx. 1 GHz comprises ultrasound.

In an example design of the ophthalmological laser therapy device, the detection system (400) includes a detector (8) for spectral detection with a detection frequency range, and is configured to visualize the detected signals of various frequencies by assigning a corresponding frequency from the visible spectrum to each frequency from the detection frequency range.

Thus, if the detection system is configured for detection of signals having different frequencies of electromagnetic examination waves from a wide frequency range, then all of the signals detected in the possible detection frequency range of the detector, for example, can be visualized, such that the entire detection frequency range of the detector is “converted” to the visible spectrum, i.e. each frequency of the detection frequency range of the detector corresponds to the visible spectrum, and a detected signal of a frequency from the invisible spectrum is depicted on a display by the frequency from the visible spectrum corresponding to this frequency.

An ophthalmological laser therapy device that includes at least one conversion layer that is placed or can be placed in the beam path of the therapy laser beam and/or in the course of the wave of the electromagnetic or mechanical examination waves is furthermore contemplated. This conversion layer is configured to convert signals of the electromagnetic examination waves from at least one frequency from the entire range of the electromagnetic examination waves into another frequency from the entire range of the electromagnetic examination waves. In particular, such a conversion layer can be configured to convert signals of the electromagnetic examination waves from at least one frequency in a frequency outside the frequency range of visible light into a signal from at least one frequency in the visible light spectrum. This also includes the possibility of the presence of a stack of different conversion layers, e.g. in order to use a larger frequency bandwidth, or in order to then be able to convert the electromagnetic examination waves of the various systems into signals in the visible light spectrum, when the electromagnetic examination waves of the various systems have frequencies that differ clearly from one another, such that they cannot all be converted by the same conversion layer into visible light.

Because a conversion layer furthermore does not normally function homogeneously over a frequency range provided for this conversion layer, it is advantageous, for example, to make a correction for this as a function of the frequency.

With such a conversion layer, or stack of different conversion layers, it is possible to convert all of the signals into the visible spectrum and visualize them collectively thereby.

Alternatively, an ophthalmological laser therapy device includes at least one scattering layer that is placed or can be placed in the beam path of the therapy laser beam and/or in the course of the waves of the electromagnetic or mechanical examination waves. This scattering layer is configured to diffuse the electromagnetic or mechanical examination waves. In particular, the scattering layer is configured for the targeted diffusion of the electromagnetic or mechanical examination waves such that, by way of example, modifications in the scattering layer generated by a therapy laser beam are made visible, and the relative positions of the therapy laser beam and electromagnetic or mechanical examination waves in relation to one another can be derived.

In addition to this possibility for the conversion of frequencies of the electromagnetic radiation of the therapy laser, or the electromagnetic or mechanical examination waves of the examination system, it is alternatively possible to work with a detector, which can detect radiation from various frequency ranges. For this, the detector can include diffractive optical elements (DOE), for example.

In order to actively adjust the signals of the therapy laser beam and the electromagnetic or mechanical examination waves in relation to one another in an observation plane of the observation volume, or in the entire observation volume, and not to merely determine the actual state, the ophthalmological laser therapy system comprises a calibration system in an example embodiment. This calibration system is configured to adjust the relative positions of the signals of the therapy laser beam and the electromagnetic or mechanical examination waves detected by the detection system in relation to one another, and to transform the coordinates from a coordinate system of the observation volume or a coordinate system of an observation plane of the observation volume to a coordinate system of the positioning system. This is possible in a two-dimensional form as long as this pertains to only one observation plane of the observation volume, but a three-dimensional view is also contemplated.

The extent of the change in the position of the signal of the therapy laser beam and electromagnetic or mechanical examination waves in the observation volume, which change is caused by the positioning system, as well as how the relationship of the signals to one another is affected, can all be determined in advance using such an ophthalmological laser therapy device. Furthermore, the actual positions of the therapy laser beam and the electromagnetic or mechanical examination waves can not only be tracked during the course of a laser therapy, but they can also be corrected. For this, an evaluation routine, to which these data are made available, can trigger an alarm, for example, and propose a correction, or automatically carry out the correction, when an internal monitoring mechanism determines that there is a deviation from the target data. Such a monitoring and correction mechanism can run in a control unit included in the calibration system, which includes a corresponding program.

As long as the ophthalmological laser therapy device includes a camera, this can be used to display the positions of the signals of the laser therapy beam and the electromagnetic or mechanical examination waves, and provide surgeons or therapists with the possibility of correcting the positions in the camera image directly, via a display input. The correction can also take place via an automatic feedback system.

The detection system of the ophthalmological laser therapy device can furthermore be configured to receive a material, for example in the observation volume or in an observation plane of the observation volume, wherein the material can be modified in a focus area of the therapy laser beam by an energy/material interaction process. Such a material is for example present in the form of a material plate, which is inserted at a position provided and prepared for this.

A calibration can be carried out particularly precisely when pre-defined, ideally three-dimensional patterns of a material received in a detection system are inscribed by application of the therapy laser beam and detected by application of the electromagnetic or mechanical examination waves are used. Such a pattern enables a coordinate transformation between a coordinate system of the observation volume, detected in the detector, and a coordinate system of the positioning system for arbitrary positions inside an observation volume to be carried out with the smallest possible deviation through an optimal selection of the sizes and positions of the structures. For this, the ophthalmological laser therapy device comprises a calibration system with a control unit, in which three-dimensional patterns for calibration are encoded such that they can be inscribed in the material that can be received in the detection system.

In an example embodiment, a three-dimensional pattern is encoded in the control unit, which comprises numerous points in different planes of the receivable material, or at least two circles in different planes of the receivable material, or lines in different planes of the receivable material. These patterns are enable quick spatial orientation with the determination of the position of the electromagnetic or mechanical examination waves in relation to the therapy laser beam. A pattern that generates at least two circles in various planes of the receivable material, when an OCT system is used, for example, enables a precise and clear determination of the positions by use of a scanning line of an OCT examination laser beam.

In order to detect and adjust the therapy laser beam and the electromagnetic examination radiation directed toward one another, a patient interface, i.e. a device normally used for determining the relative position of an eye to the ophthalmological laser therapy device, can also be implemented in an ophthalmological laser therapy device, as long as options for this are provided on the patient interface.

A patient interface for an ophthalmological therapy device, in particular for an ophthalmological laser therapy device, therefore includes features for determining and for the collective depiction of the relative positions of the signals of electromagnetic and/or mechanical waves of different frequencies to one another, in particular features for determining the positions of the therapy laser beam and electromagnetic or mechanical examination waves.

In an advantageous example embodiment, the patient interface includes features for determining the positions of signals of electromagnetic and/or mechanical waves of different frequencies on a protective cap, with which the patient interface is normally sealed prior to use, in order to keep it sterilized.

Such a protective cap can also be formed by a simple film as well. This allows, for example, a simple and precise detection of the positions of the therapy laser beam and signals of the electromagnetic or mechanical examination waves prior to starting the therapy, i.e. before the system is applied to a patient's eye. Such a protective cap, or film, can thus not be implemented during the therapy for detection and calibration: During the therapy, it is then possible, however, to further work, for example, with structures on the patient interface itself, or structures that are located in the ophthalmological laser therapy system that can be inserted into the beam path, or by using the structures of the eye.

In particular, a patient interface can include a conversion layer or a scattering layer. Such a conversion layer or scattering layer can be applied to the protective cap or film of the patient interface, or directly to the patient interface. Both variations can also be used simultaneously thereby, such that first, e.g. prior to starting an ophthalmological laser therapy treatment, the layer (or layers) of the protective cap can be used for calibration, while a layer applied directly to the patient interface is merely registered. In contrast, with subsequent controls and corrections during the therapy phase, the layer or layers applied directly to the patient interface are used.

A patient interface can also include a region having a material that can be modified in a focus area of the therapy laser beam by an energy/material interaction process. This material, which can be modified by the therapy laser beam, can be applied to the protective cap of a patient interface, or directly on the patient interface. The use of the patient interface for the detection and calibration of the positions of signals of electromagnetic and/or mechanical waves of different frequencies, in particular the positions the therapy laser beam and electromagnetic or mechanical examination waves has the advantage that for the respective individual therapy procedure, a calibration can be carried out on the disposable material necessarily used for this procedure.

Moreover, the patient interface offers specifically a possibility, on one hand, for depicting the positions of the signals in a collective reference system, and thus to establish a relationship between the two, and on the other hand, to simultaneously also establish the relative positions of the eye to be treated to the ophthalmological laser therapy device, and thus to the signals of the therapy laser beam and the electromagnetic or mechanical examination waves.

In a calibration method according to the invention, for an ophthalmological laser therapy device described above, the therapy laser beam having a first frequency and the electromagnetic or mechanical examination waves having a second frequency are successively moved or displaced laterally, in two different directions, to a specific extent, in a first step, and the respective coordinates of the positioning system that is responsible for the displacement and positioning of the laser therapy beam and the electromagnetic or mechanical examination waves, as well as the signals from the therapy laser beam and electromagnetic or mechanical examination waves are determined in the observation volume, this first step is then repeated at least once at another location, and the coordinates of the positioning system are allocated to the respective coordinates of the signals from the therapy laser beam and electromagnetic or mechanical examination waves in the observation volume or in an observation plane of the observation volume of the detection system. The respective coordinates of the positioning system—either separately for the therapy laser beam and the electromagnetic or mechanical examination waves, as long as the control unit can cause their deflections independently, or collectively, as long as a collective deflection takes place—are then assigned to the values of a grid system in the observation image of the observation volume of the detection system. With a fixed relationship between the therapy laser beam and the electromagnetic or mechanical examination waves, e.g. an OCT examination laser beam, the spacing between the two beams, or the two signals can thus be determined, and provided as a fixed offset.

It is advantageous thereby, for example, when in the calibration procedure, for lateral calibration of the therapy laser beam and/or the electromagnetic or mechanical examination waves, the profile of the therapy laser beam or electromagnetic or mechanical examination waves is determined through calculating a “comparison image” in each case, i.e. an observation volume is detected with and without a therapy laser beam or with and without electromagnetic or mechanical examination waves, and the differences between the two generated images are determined. The respective focal point of the beam can then be determined from the profile.

Alternatively, instead of a comparison image, a template of a laser signal of a therapy laser, as well as signals of the electromagnetic or mechanical examination waves, can be used, and the currently generated observation image in the observation volume can be compared with the template by application of an algorithmic comparison, thus a template-supported image recognition can be used.

Furthermore, it is advantageous, for example, when in the calibration procedure for axial calibration, the focal position of the therapy laser beam and/or the electromagnetic or mechanical examination waves, as long as these are focused, is changed in the axial direction and the intensity and shape of the respective signals are evaluated thereby.

If a material, in particular a material plate, is inserted in the detection system during the calibration procedure, a modification in the material can be caused in the focus area of the therapy laser beam having a first frequency through an energy/material interaction process, and this can be detected using electromagnetic or mechanical examination waves of the examination system having a second frequency and/or using the electromagnetic waves of the camera system having a third frequency from the visible spectrum, for example in a visual recording.

The causing of a modification in the material leads to a lasting marking of the signal position of a therapy laser beam, which then makes it no longer necessary to detect the signal of the therapy laser beam itself, but rather, the detection of the material modification can be used for determining the position.

It is particularly advantageous, for example, when in such a calibration procedure, a predefined, for example three-dimensional pattern can be formed in the material by application of the energy/material interaction process. This pattern should then be encoded such that the structural sizes of the structures of the pattern and the configuration of the structures contributes to the determination of the positions of the signals of the therapy laser beam and the electromagnetic or mechanical examination waves with the highest possible precision.

In an ophthalmological laser therapy method according to the invention, the positions of the signals of the therapy laser beam and the electromagnetic or mechanical examination waves in relation to one another are set by a calibration procedure. The coordinate data of the detection system and the positioning system determined thereby are stored during the calibration procedure, and subsequently used for positioning the therapy laser beam and the electromagnetic or mechanical examination waves during the laser therapy treatment.

It is facilitated by the devices and methods described herein that a clear correlation can be established between the therapy laser beam, the electromagnetic or mechanical examination waves of an examination system, for example an OCT system or an alternative method such as an ultrasound imaging process, and, if applicable, a visual recording of the eye, which can be used for the ophthalmological laser therapy method. In addition, it is ensured that the correlation is verified during the course of a laser therapy method, and can be corrected at any time, if necessary. The examination system, such as an OCT system, for example, as well as optical recordings, if applicable, can be used, repeated and corrected, in order to precisely carry out the planned incision in the eye.

### BRIEF DESCRIPTION OF THE DRAWINGS

The present invention shall now be explained on the basis of example embodiments:

FIG. 1 is a simplified schematic illustration of a first ophthalmological laser therapy device as well as an optical path in this laser therapy device.

FIGS. 2A and 2B depict a first and second variation of a patient interface according to the invention: FIG. 2a illustrates a patient interface with a protective cap having an integrated scattering layer or conversion layer, while FIG. 2b shows a patient interface with a scattering layer or conversion layer in a sterile cover of the patient interface.

FIG. 3 is a simplified schematic illustration of the optical path in a third laser therapy device according to the invention.

FIGS. 4A, 4B, 4C depict patterns for calibrating the position of an OCT laser beam and therapy laser beam, as well as, if applicable, the visual recording in the visible light spectrum, in each case in a top view and a side view.

FIG. 5 depicts an exemplary embodiment of a patient interface, with which the positions can be monitored prior to and during each operation. Thereby, FIG. 5a shows the variation of a contact lens, on the edge of which a detection layer is applied, while FIG. 5b shows the variation of a patient interface having a detection layer ring on the inside of a funnel.

FIGS. 6A and 6B depict the calibration or control of the calibration of the therapy laser beam and OCT laser beam by use of a spot on a contact lens.

FIGS. 7A and 7B depict patterns for a calibration of the visual recording in the visible light spectrum with the OCT laser beam.

### DETAILED DESCRIPTION

There are three fundamental possibilities to automatically determine, monitor, and, if applicable, calibrate the focal positions of the therapy laser beam and the OCT laser and/or other focused electromagnetic examination radiation: The non-invasive methods, invasive methods, and indirect methods, which shall be described in detail below.

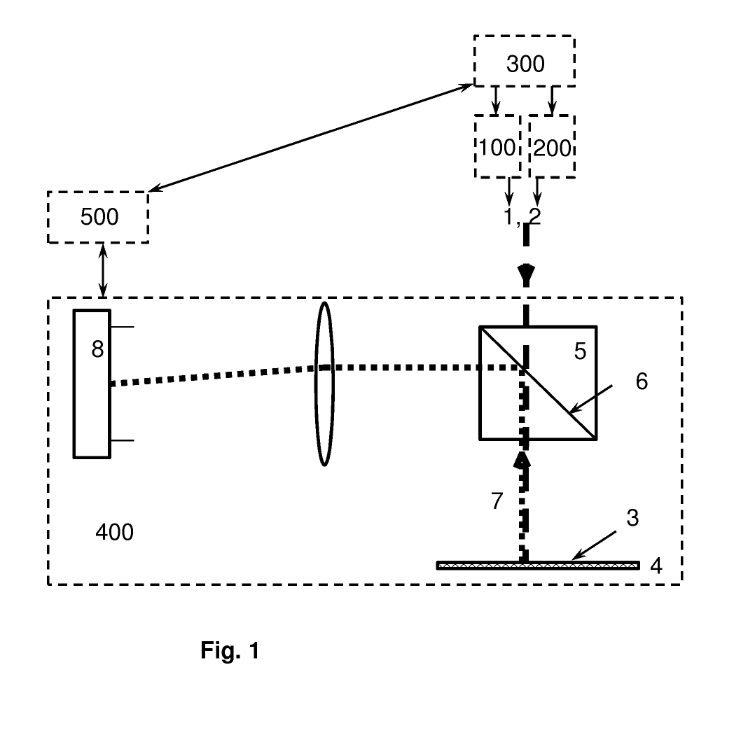

In a first ophthalmological laser therapy device according to an example embodiment of the invention, a non-invasive method is used in conjunction with various detection methods. FIG. 1 shows a simplified schematic illustration of a first ophthalmological laser therapy device, having a laser system 100, an examination system 200, a positioning system 300, a detection system 400, and a calibration system 500. It also illustrates the optical system of such an ophthalmological laser therapy device, and thus the optical path in this first ophthalmological laser therapy device according to the invention, in which an OCT system is used as the examination system 200. Furthermore, there is a camera system 8, which generates visual recordings in the visible spectrum. Visual recordings in the context of this invention are always to be regarded as visual recordings or images created in the visible spectrum.

In this case, the visual recordings are used for detection of the therapy laser beam 1 and the OCT laser beam 2. For the detection and calibration, three-dimensional focal positions of the therapy laser beam 1 and the position of the OCT laser beam 2, both of which lie in the near infrared radiation (NIR) or infrared radiation (IR) frequency range, are determined in the visual recording, i.e. the camera image. Because the visual recordings can only be generated with the camera system 8 in this first ophthalmological laser therapy device with visible light, a material is necessary, which converts the near infrared radiation (NIR) or the infrared radiation (IR) from the therapy laser beam 1 and OCT laser beam 2 to the visible range. Such a conversion material, or such a conversion layer 3, is available commercially for various input frequencies that are to then be converted to the visible spectrum. In order to image the focal positions of the therapy laser beam 1 and the OCT laser beam 2 in the visual recording, a conversion layer 3 is inserted in an observation plane 4 of an observation volume 9, or at the entry of an observation volume 4. This converts the near infrared radiation (NIR) or infrared radiation (IR) of the therapy laser beam 1 or the OCT laser beam 2 into visible light. The OCT laser beam 2 and therapy laser beam 1 then first pass though a beam splitter 5 in the ophthalmological laser therapy device according to the invention, without being affected by its reflection layer 6. The conversion layer 3 on the observation plane 4 converts the near infrared or infrared radiation of the OCT laser beam 2, or the therapy laser beam 1 into the visible spectrum. This visible light is reflected by the reflection layer 6 of the beam splitter, and deflected to the detection system. As a result, the therapy laser beam 1 and OCT laser beam 2 can be imaged in the visual recording. The visual recording can thus be used directly for determining an offset of the therapy laser beam 1 and the OCT laser beam 2, and taken into account in the further course of the procedure. In addition, the position of the therapy laser beam 1 and the OCT laser beam 2 can be determined inside the visual recording of the camera system 8, with which the eye of a patient is later imaged, wherein the position of the incision in the eye is established in this recording.

Alternatively, a second ophthalmological laser therapy device according to the invention, for a non-invasive method, is designed such that the wavelengths, and thus the frequencies, of the therapy laser beam 1 and the OCT laser beam 2 from the frequency range of the near infrared radiation (NIR) or infrared radiation (IR) are also deflected in part to a detector 8 and detected there, and can thus be made visible. Such a deflection is possible, for example, through the use of a beam splitter, which reflects a small percentage of the light. With the second ophthalmological laser therapy device according to the invention, in comparison to the first ophthalmological laser therapy device according to the invention, no frequency conversion is necessary, and an arbitrary scattering layer or reflection layer can be used in place of the conversion layer 3. The layer is inserted in the observation plane 4 of an observation volume 9 of the optical system of the second ophthalmological laser therapy device according to the invention. Such a second ophthalmological laser therapy device according to the invention substantially corresponds to the first ophthalmological laser therapy device according to the invention from FIG. 1 in terms of the configuration of its optical elements, but instead of a conversion layer 3, there is a scattering layer or reflection layer in the beam path. The detector 8 in this second ophthalmological laser therapy device according to the invention enables a spectral detection over the frequency range from infrared radiation to the near ultraviolet radiation range.

In order to then make the detected frequencies from the entire detectable frequency range visible for an operator or user of this second ophthalmological laser therapy device according to the invention, the entire detectable frequency range of the detector 8 is mapped on a visible light spectrum, and depicted in the corresponding colors of visible light. For this, the detector 8 includes a microcontroller and a display (neither of which are shown in FIG. 1), wherein the spatially resolved imaging of the respected detected frequencies takes place in the corresponding imaging colors on the display.

In order to detect the focal positions of the therapy laser beam 1 and the OCT laser beam 2, a first image is recorded without laser signals—in the form of a visual recording or by a detector 8, which can spectrally detect in a spatially resolved manner, thus able to detect the striking of radiation of different frequencies from a wide frequency range in a spatially resolved manner, including electromagnetic radiation in the near infrared range or infrared range. The therapy laser and the OCT laser are then successively switched on, and an image is then recorded. The beams can be registered in the images as light points with the use of the conversion layer 3 or scattering layer.

The lateral detection and calibration takes place in the following manner: The therapy laser beam 1 or the OCT laser beam 2 can be detected directly in the image; in the case of the first ophthalmological laser device according to the invention, in the visual recording of the camera system. For this, a template of the laser signal can first be generated. This template is then used to locate the signal of the therapy laser beam 1 or the OCT laser beam 2 in an image recorded later, through an algorithmic comparison of the template with the image contents of the image recorded later of the therapy laser beam 1 or the OCT laser beam 2.

Alternatively, a comparison image can also be calculated from an image without, and an image with, laser signals 1, 2. The positions of the laser beams 1, 2 can be determined in the comparison image, for example, in that the region having the highest intensity is first determined. Subsequently, the profile of the laser beam 1, 2 is then adjusted to this value, and the focal point of the laser beam is determined. In this manner, the precise position and the intensity distribution can be determined for this position in the image, i.e. in the visual recording in the case of the first ophthalmological laser therapy device according to the invention. In this manner, it is thus possible to determine the lateral positions of the OCT laser beam 2 and the therapy laser beam 1 in relation to one another and in the visual recording.

The calibration in the axial direction, i.e. perpendicular to the visual recording, or to the detector 8, respectively, takes place by changing the focal positions of the two laser beams in this direction. By evaluating the intensity and shape of the signal in each position, in particular the diameter, the focal position can be adjusted such that the focus lies in the observation plane 4 of the visual recording. This is then achieved when the signal has the highest intensity with the smallest diameter.

By establishing the axial focal position of the one beam, e.g. the OCT laser beam 2, and the adjustment of the axial focal position of the other beam, e.g. the therapy laser beam 1, both beams can be brought into an observation plane 4. As a result, the lateral position of the therapy laser beam 1 in relation to the OCT laser beam 2 and to the visual recording, as well as the OCT laser beam 2 in relation to the visual recording, is known thereby, and the axial focal positions are set to the observation plane 4, and thus likewise known.

The adjustment of the positions of the laser beams from the therapy laser beam 1 and the OCT laser beam 2 normally takes place automatically. The positions of the laser beams can be adjusted thereby, e.g. through a moved or tilted mirror or lens, or through movement of the beam source. For this, the offset of such a movement or tilting in the observation plane 4 must be known. In order to determine the coordinate transformation between a positioning system 300 of the laser beams, thus, e.g., the moved or tilted mirrors or lenses, or the movement of the laser beam source, respectively, and the positions of the laser beams in the visual image, which serves here as a visual co-observation image, the therapy laser beam 1 and the OCT laser beam 2 are successively moved laterally in two different directions to a specific extent. This procedure is repeated for numerous points, or for at least two points. The calibration becomes increasingly precise as more points are used here. The positions of the positioning system 300, thus the displacing system, e.g. the moved or tilted mirrors or lenses, or the movement of the beam source, are then assigned values in a grid system in the observation volume 9 of the detection system 400. Thus, a relationship between the set values of the displacing system and the positions of the laser beams in the observation plane 4 or in the observation volume 9, can be established, such that a transformation of the coordinates of both systems into one another is possible. If the therapy laser beam 1 and the OCT laser beam 2 have a fixed local relationship to one another, as is the case, for example, when they are displaced with the same displacing system, then the spacing can be determined with this method, and provided as a fixed offset in the overall system. In this manner, it is ensured that the measurements of the examination procedure, in this case the imaging OCT procedure, take place at the location where the therapy laser makes an incision. in a special design, a variable offset for a spacing that changes as a function of the position of the displacing system would also be conceivable.

If the orientation is to be checked prior to the ophthalmological laser therapy, the use of a special patient interface 10 according to the invention is possible in another ophthalmological laser therapy device according to the invention:

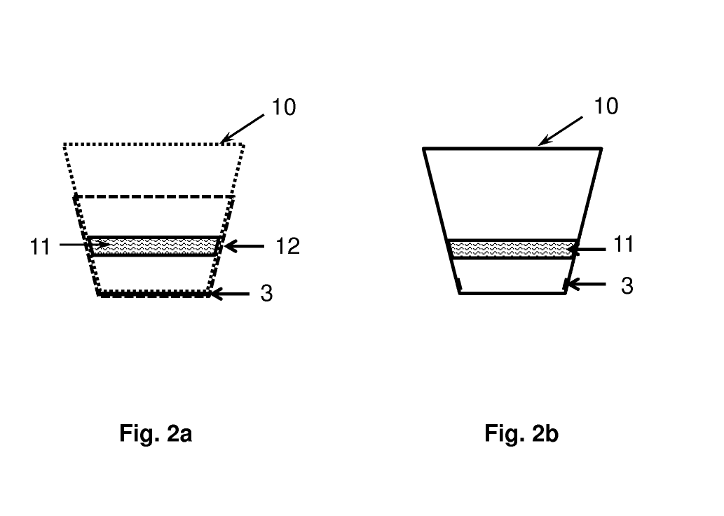

Shortly before the laser treatment, the optical system of the ophthalmological laser therapy device is mechanically and optically coupled to the patient eye by use of a patient interface 10. The patient interface 10 usually comprises a frame with a lens, the so-called contact lens 11. In order to keep the patient interface 10 sterile, it is sealed with a protective cap 12 or a protective film. A patient interface 10 according to the invention is thus sealed in a first variation with a protective cap 12, which is coated with a conversion layer 3. This is illustrated in FIG. 2a. In a second variation, illustrated in FIG. 2b, the patient interface 10 is provided with a protective film as a sterile cover, which comprises a conversion layer 3. The position and expansion of the conversion layer 3 on the protective cap 12 or the protective film can be adapted thereto on an individual basis.

The protective cap 12, or the protective film is impermeable for the selected wavelength spectrum of the therapy laser beam 1 as well as the OCT laser beam 2. The positions of the laser beams are monitored with the method described above. Thus, the positions of the laser beams are determined with either a comparison image comprising a comparison between a switched-on and a switched-off state of the laser in question, or between a template and the actual image of the laser, or they are determined with another image processing method. The positions of the laser beams are determined thereby, at least at one point, and compared with the predefined positions. If the positions are aligned, the protective cap 12 or the protective film can be removed, the patient interface 10 applied to the eye, and the treatment started. If the positions are not aligned, the calibration is executed automatically. Numerous predefined positions are set using the positioning system 300, and the positions determined in the observation plane 4. As a result, a new coordinate transformation can be calculated. The procedure can be executed for different axial planes.

In a modification of the first variation as well as the second variation of the patient interface 10 according to the invention, the protective cap 12, or the protective film, is coated with a scattering layer instead of a conversion layer 3.

The known positions and the knowledge regarding the coordinate transformation between the coordinate system of the observation plane 4 or the observation volume 9 and the coordinate system of the positioning system 300, which includes, for example, a mirror, then serve, for laser treatment in the eye, as a basis for approaching and imaging specific positions, or to be able to trace corresponding treatment patterns during the therapy procedure with the therapy laser beam 1 and the OCT laser beam 2. The respective positions are defined thereby in the observation volumes 9 or in an observation plane 4 of the observation volume 9, which are additionally observed in a visual recording. The coordinates are then transformed into the mirror coordinate system, and the mirror is moved accordingly, such that the OCT laser beam 2 arrives at the desired position. The therapy laser beam 1 is then adjusted in the same manner.

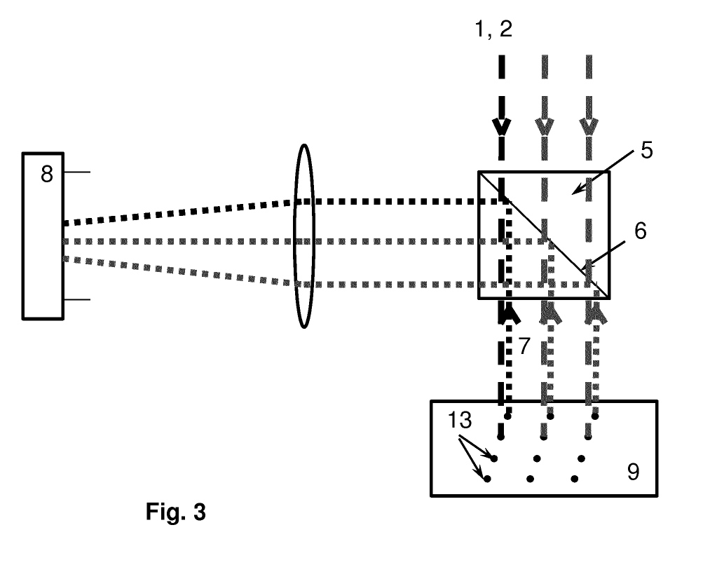

An invasive method in a third ophthalmological laser therapy device according to the invention, shall now be described, which can be combined with various detection methods: One such possibility for determining the focal position of the therapy laser beam 1 and the OCT laser beam 2 in relation to the visual recording in the visible spectrum is composed of generating a modification in a material with the laser beam 1. For this, a material plate is inserted in the observation plane 4 of the detection system 400. PMMA, glass, etc. are materials suitable for this. A modification in the material is caused by of the therapy laser beam 1 in its focus area by an energy/material interaction process. This is illustrated in FIG. 3, which shows a simplified schematic depiction of the optical path in such a third laser therapy device according to the invention. Here as well, the OCT laser beam 2 and the therapy laser beam 1 pass through a beam splitter 5, without the influence of a reflection layer 6 located therein, and strike the material plate, or a material block. The therapy laser beam 1 generates a defined pattern 13 in the material block through a material modification by 79 use of sufficiently high energy. In a visual recording 8 used for detection, only the pattern plane that lies in the corresponding observation plane 4 of the observation volume 9 is visible.

The laser radiation induces local modifications, such as a local expansion or a generation of gas bubbles, through a sufficiently high energy in such an invasive procedure, which can be detected using a visual recording in the visible spectrum, or with other detectors 8, or detection methods. The laser power is selected in invasive methods, such that the smallest acceptable detectable modifications are generated, in order to obtain the highest correlation precision. The position of the therapy laser beam 1 can be determined using image processing algorithms or comparison images, in this case illustrating the differences between an image without laser induced modifications and an image with laser induced modifications.

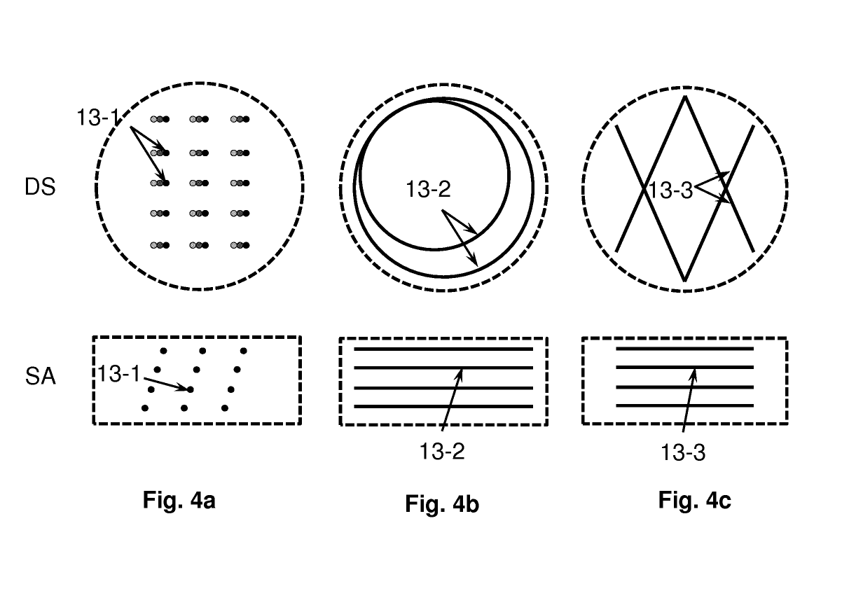

While the position of the therapy laser beam 1 in the observation pane 4 can now be determined through the incorporation of a pattern point, a predefined pattern 13, which includes more than one point, must be formed in the material for adjusting the therapy laser beam 1 to the OCT laser beam 2. Possible solutions for such a pattern 13 for calibrating the positions of the OCT laser beam 2 and the therapy laser beam 1, as well as, if applicable, the visual recording 8 in the visible light spectrum, are, e.g. a 3D point pattern, numerous lines, or two inscribed, non-concentric circles, as is shown in FIGS. 4a to 4c, in both a top view DS and a side view SA.

For the first pattern 13-1 shown in FIG. 4a, points are formed in different observation planes 4. For the second pattern 13-2 shown in FIG. 4b, two circles are formed in different observation planes 4, and for the third pattern 13-3 shown in FIG. 4c, lines are formed in different observation planes 4. Through the use of numerous observation planes in which the pattern 13 is formed from the bottom up, and after the formation of the pattern 13, the respective observation planes 4 are examined with the OCT laser beam 2 and detected, e.g., in a visual recording 8, there is no need for prior knowledge regarding the axial focal position of the therapy laser beam 1.

A line-scan of the second pattern 13-2 shown in FIG. 4b with an OCT laser beam 2 makes it possible to precisely and clearly determine the position of the scanning line. With the pattern 13-3 shown in FIG. 4c, two line-scans are needed. For the second and third patterns 13-2, 13-3, the position of the scanning line in relation to the pattern 13 can be determined form the spacing of the detected points in the line-scan. Using this information, the coordinate transformation between the visual recording 8, the therapy laser beam 1, and the OCT laser beam 2 can be calculated in two dimensions. If a pattern 13 having different axial planes is generated by the laser/material interaction, then the structure only appears to be in sharp focus in the axial plane that is aligned with the observation plane 4 of the optical observation. Thus, the axial focal position is set to the observation plane 4. A further possibility for determining the axial position of the pattern 13 is the use of an OCT image. As a third variation, a confocal detection system may additionally be provided in the system. If a reflecting layer, e.g. a glass plate, is placed in the beam path, and moved axially through a focus, a strong signal form the confocal detector is obtained in the focal plane, and the focal position is thus determined. Alternatively, the focal position can also be displaced in the axial direction.

The monitoring and automated correction of the position of the therapy laser beam 1 and an OCT laser beam 2 takes place analogously to the monitoring and automated correction in the non-invasive method described above. However, with the invasive method, structural modifications are formed in a material located in the beam path, such as in the cover of the patient interface 10, thus the protective cap 12 or the protective film, or directly in the patient interface 10.

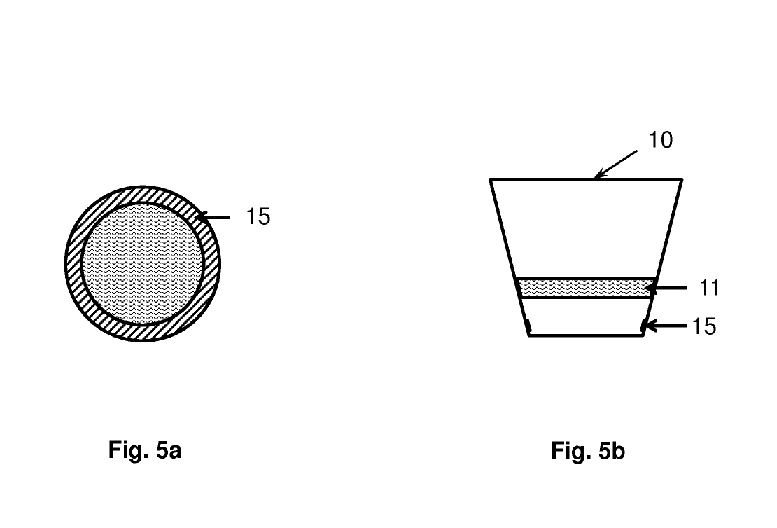

For example, a “sacrifice layer,” thus a possibility of a detection layer 15, that can be modified through an energy/material interaction process, can be placed on the edge of the patient interface 10. A monitoring of the positions of this type, prior to each operation, by use of such a detection layer 15 is illustrated in FIGS. 5a and 5b. A detection layer 15 can be applied on the edge of a patient interface 10, as shown in FIGS. 5a and 5b, in the same manner, when this is a conversion layer or a scattering layer instead of a sacrifice layer. FIG. 5a shows a variation of a contact lens 11, on the edge of which a detection layer 15 is applied, while FIG. 5b shows the variation of a patient interface 10 with a detection layer ring 15 on the inside of the funnel, thus the frame of the patient interface 10. The use of a detection layer 15 on the edge of a contact lens 11 is only possible, however, when the observation plane 4 in the observation volume 9 can be displaced during the detection, because the contact lens plane would otherwise not be in focus. When a detection layer 15 is used on the inside of the funnel of the patient interface 10, it is possible to place the ring in a fixed observation plane 4 of the observation volume 9. This type of monitoring is then only possible, however, when the observation field of the detector, e.g. the camera system 8 having the visual recording, is sufficiently large.

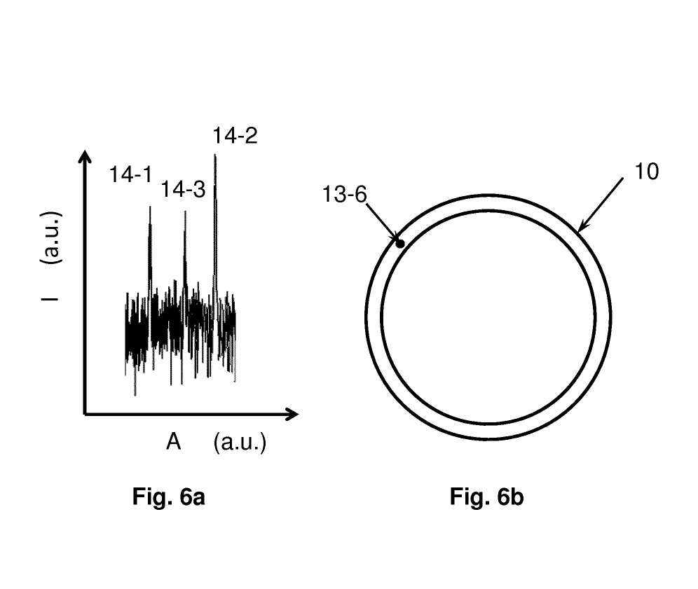

The therapy laser beam 1 and the OCT laser beam 2 are set to a specific position, and the positions of the two beams are determined, as described above, in the visual recording, for example. In addition, a spot 13-6, or a point, can be etched into the edge of the contact lens 11, as is shown in FIG. 6b, which is detected with the OCT laser beam 2. In this manner, the axial focal position of the therapy laser beam 1 can be checked. Alternatively, a conversion layer 3, as described in reference to the first method, can also be used.

The calibration or monitoring of the calibration of the therapy laser beam 1 and the OCT laser beam 2 by forming a spot 13-6 in the edge of the contact lens is shown in FIG. 6. The material modification is detected with the OCT laser beam 2. FIG. 6a shows an excerpt of an A-scan. Three peaks 14-1, 14-2, 14-3 rise above the background noise. The two outer peaks 14-1, 14-2 indicate the upper and lower surfaces of the contact lens 11. The peak 14-3 in the middle indicates the material modification.

An indirect method for calibrating an OCT laser beam 2, a therapy laser beam 1, and a visual recording 8 to one another through the use of visible light, using a fourth ophthalmological laser therapy device according to the invention, shall now be described.

First, the position of the OCT laser beam 2 is determined in a visual recording 8. Subsequently, the position of the therapy laser beam 1 is determined in relation to the OCT laser beam 2, by which the position of the therapy laser beam 1 is also defined in the visual recording 8. In order to determine the position of the OCT laser beam 2 in the visual recording 8, a pattern 13 that can be detected by the OCT system and is visible in the visual recording 8 is inscribed in an observation plane 4 of the observation volume 9 of the system. A substrate serves as a medium for the pattern 13, wherein the pattern 13 that is applied to the substrate must reflect or scatter more strongly than the substrate itself. By way of example, glass may be used as the substrate, when the layer of the pattern 13 is made of gold or plastic, in order to be detected with an OCT system.

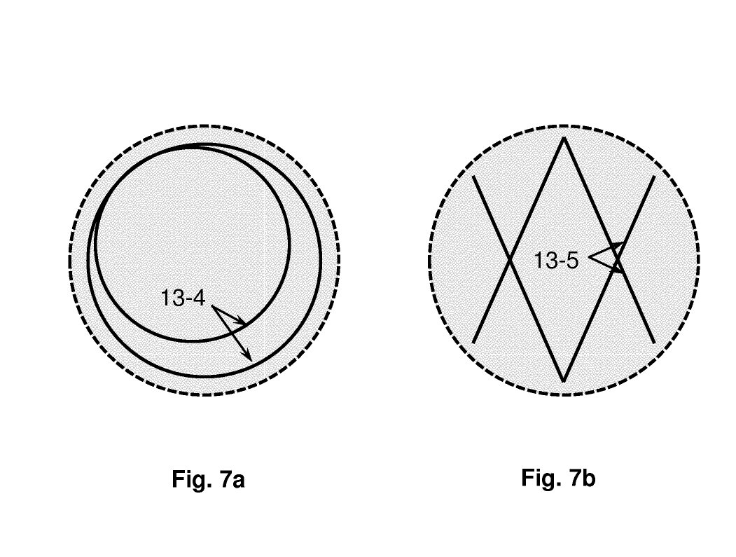

Possible designs of a pattern 13-4, 13-5 for the calibration of the visual recording in the visible light spectrum with the OCT laser beam 2 are shown in FIGS. 7a and 7b. Although the pattern 13-5 in FIG. 7b must be attached in a defined rotational direction, the pattern 13-4 in FIG. 7a offers the advantage that the stops of the moving system can be determined, for which reason the direction of rotation during the installation of the pattern 13-4 can be selected freely.

The patterns 13-4, 13-5 can be detected in the visual recordings and processed by using software, such that the precise position and the orientation of the pattern 13-4, 13-5 in relation to the visual recording 8 is known. First, two parallel line-scans are executed with the OCT system at different positions. The pattern 13-4, 13-5 is detected along this line, such that the positions of the displacing system, thus the positioning system 300, can be assigned to the patterns 13-4, 13-5, and thus to the positions in the visual recording 8. The second line scan is used to clearly determine the orientation of the pattern 13-4, 13-5 in relation to the positioning system of the OCT laser beam 2.

A spot 13-6 can then be etched into a region of a contact lens 11 secured on the ophthalmological laser therapy device that is not used optically. The spot 13-6 is thus located in a defined lateral and axial position. This spot 13-6 is then detected with the OCT system, and registered. A scanning over a surface region may be necessary for this. If the etched spot 13-6 is detected, the lateral offset of the therapy laser beam 1 and the OCT laser beam 2 can be determined from the different positions of the positioning system at the time of the etching and at the time of the detection. Because the position of the OCT laser beam 2 has already been clearly determined inside the visual recording 8, the position of the therapy laser beam 1 is also clearly defined in the visual recording 8 via the correlation of the OCT laser beam 2 and the therapy laser beam 1. In addition, the axial focal position can also be checked during the detection of the spot 13-6 with the OCT system.

Another possibility for checking the axial focal position is the additional use of a confocal detection system.

The focal position can be determined or checked in the manner described in the last section of the example of the invasive method.

The features specified above and explained in reference to various exemplary embodiments can be used not only in the combinations given by way of example, but also in other combinations, or in and of themselves, without abandoning the scope of the invention.

A description referring to a device feature applies analogously with respect to this feature to the corresponding method, while method features represent corresponding functional features of the invention.

1.-22. (canceled)

23. An ophthalmological laser therapy device comprising
a laser system that generates a therapy laser beam having a first frequency to treat eye tissue;
an examination system that obtains data regarding the structure of the eye by electromagnetic or mechanical examination waves having a second frequency;
a positioning system that controls the therapy laser beam and the electromagnetic or mechanical examination waves;
a detection system, comprising a detector and an observation volume, and that detects and collectively depicts, in a repeatable, spatially resolved manner, signals of the therapy laser beam and the electromagnetic or mechanical examination waves striking an observation plane of the observation volume or in the entire observation volume, and relative calibration thereof.

24. The ophthalmological laser therapy device according to claim 23, further comprising a detection system, configured to determine comparison images and/or for template supported image recognition.

25. The ophthalmological laser therapy device according to claim 23, further comprising a detection system, which comprises a camera system, that generates a visual recording of structures of the eye by capturing electromagnetic waves having a third frequency from the visible spectrum, and that detects spatially resolved signals and collectively depicts the signals striking an observation plane of an observation volume or in the entire observation volume, and depicts signals of the therapy laser beam and/or the electromagnetic or mechanical examination waves.

26. The ophthalmological laser therapy device according to claim 23, further comprising a detection system that detects signals having different frequencies from electromagnetic examination waves in a frequency ranging from microwaves to X-ray radiation

26. The ophthalmological laser therapy device according to claim 26, wherein the detection system detects signals in a frequency range from infrared light to the entire visible spectrum, detects mechanical examination waves in the ultrasound frequency range or both of the foregoing.

26. The ophthalmological laser therapy device according to claim 26, further comprising a detection system, comprising a detector for spectral detection with a detection frequency range, and which is configured to make the detected signals of various frequencies visible by assigning a corresponding frequency from the visible spectrum to each frequency in the detection frequency range.

29. The ophthalmological laser therapy device according to claim 23, further comprising at least one conversion layer that is or can be inserted in a beam path or wave course of the therapy laser beam, the electromagnetic or mechanical examination waves or both of the foregoing, that converts signals from at least one frequency of the electromagnetic examination waves into another frequency of the electromagnetic examination waves or a scattering layer that scatters the electromagnetic or mechanical examination waves.

30. The ophthalmological laser therapy device according to claim 29, wherein the at least one conversion layer converts at least one frequency from the frequency range lying outside the visible light frequency, into signals having frequencies from the visible light frequency.

31. The ophthalmological laser therapy device according to claim 23, wherein the examination system comprises an optical coherence tomography system, that generates electromagnetic examination waves in the form of an examination laser beam.

32. The ophthalmological laser therapy device according to claim 31, wherein the optical coherence tomography system generates focused electromagnetic waves in the form of a focused examination laser beam.

33. The ophthalmological laser therapy device according to claim 23, further comprising a calibration system that adjusts a relative position of the signals detected by the detection system to one another, and for coordinate transformation between a coordinate system of the observation volume and a coordinate system of the positioning system.

34. The ophthalmological laser therapy device according to claim 23, wherein the detection system is configured to receive a material, which can be modified in a focus area of the therapy laser beam by an energy/material interaction process.

35. The ophthalmological laser therapy device according to claim 34, further comprising a calibration system, which comprises a control unit, in which a three-dimensional pattern is encoded for calibration, such that the three-dimensional pattern can be inscribed in a material that can be received in the detection system.

36. The ophthalmological laser therapy device according to claim 35, wherein the control unit, in which a three-dimensional pattern that is encoded, comprises numerous points in different planes of the receivable material, or at least two circles in different planes of the receivable material, or lines in different planes of the receivable material.

37. The ophthalmological laser therapy device according to claim 23, further comprising a patient interface, the patient interface comprising means for determining, and the collective depiction of, the relative positions of signals of the therapy laser beam and electromagnetic and/or mechanical examination waves of different frequencies in relation to one another.

38. The ophthalmological laser therapy device according to claim 37, wherein the means for determining, and for the collective depiction of, the relative position are located in a protective cap, with which the patient interface is sealed.

39. The ophthalmological laser therapy device according to claim 37, wherein the patient interface further comprises a conversion layer or scattering layer.

40. The ophthalmological laser therapy device according to claim 37, wherein the patient interface further comprises a region having a material that can be modified in a focus area of the therapy laser beam by an energy/material interaction process.

41. A patient interface for an ophthalmological therapy device, comprising means for determining, and the collective depiction of the relative positions of signals of the therapy laser beam and electromagnetic and/or mechanical examination waves of different frequencies in relation to one another.

42. The patient interface according to claim 41, wherein the means for determining, and for the collective depiction of, the relative position are located in a protective cap, with which the patient interface is sealed.

43. The patient interface according to claim 41, further comprising a conversion layer or scattering layer.

44. The patient interface according to one of the claims 41, further comprising a region having a material that can be modified in a focus area of the therapy laser beam by an energy/material interaction process.

45. A calibration method for an ophthalmological laser therapy, comprising:
in a first step, successively laterally moving a therapy laser beam having a first frequency, and the electromagnetic or mechanical examination waves having a second frequency, in two different directions to a specific extent, and determining respective coordinates of a positioning system and signals from the therapy laser beam and the electromagnetic or mechanical examination waves in the observation volume;
repeating the first step at least once at another location;
allocating coordinates of the positioning system to respective coordinates of signals from the therapy laser beam and the electromagnetic or mechanical examination waves in observation volumes of the detection system.

46. The calibration method according to claim 45, further comprising, for the lateral calibration of the therapy laser beam and/or the electromagnetic or mechanical examination waves, determining a profile of the therapy laser beam, or the electromagnetic or mechanical examination waves by calculating, in each case, a comparison image, by an algorithmic comparison or both of the foregoing.

47. The calibration method according to claim 45, further comprising, for the axial calibration, changing the focal position of the therapy laser beam and/or the electromagnetic or mechanical examination waves in an axial direction, and evaluating an intensity and shape of a respective signal.

48. The calibration method according to claim 45, further comprising inserting a material in the detection system, modifying the material in a focus area of the therapy laser beam by an energy/material interaction process, and detecting this modification with the electromagnetic or mechanical examination waves of the examination system, with the electromagnetic waves of the camera system having a third frequency from the visible spectrum or both of the foregoing.

49. The calibration method according to claim 48, further comprising detecting the modification in a visual recording.

50. The calibration method according to claim 48, further comprising forming a predefined pattern in the material by the energy/material interaction process.

51. An ophthalmological laser therapy method, comprising:
adjusting positions of signals of a therapy laser beam and electromagnetic or mechanical examination waves in relation to one another by a calibration method, comprising
in a first step, successively laterally moving a therapy laser beam having a first frequency, and the electromagnetic or mechanical examination waves having a second frequency, in two different directions to a specific extent, and determining respective coordinates of a positioning system and signals from the therapy laser beam and the electromagnetic or mechanical examination waves in the observation volume;
repeating the first step at least once at another location;
allocating coordinates of the positioning system to respective coordinates of signals from the therapy laser beam and the electromagnetic or mechanical examination waves in observation volumes of the detection system; and
the therapy method further comprising storing the coordinate data of the detection system and the positioning system thereby, and using the coordinate data of the detection system and the positioning system to position the therapy laser beam and the electromagnetic or mechanical examination waves during the laser therapy treatment.

52. The ophthalmological laser therapy method according to claim 51, wherein the calibration method further comprises, for the lateral calibration of the therapy laser beam and/or the electromagnetic or mechanical examination waves, determining a profile of the therapy laser beam, or the electromagnetic or mechanical examination waves by calculating, in each case, a comparison image, by an algorithmic comparison or both of the foregoing.

51. The ophthalmological laser therapy method according to claim 51, wherein the calibration method further comprises, for the axial calibration, changing the focal position of the therapy laser beam and/or the electromagnetic or mechanical examination waves in an axial direction, and evaluating an intensity and shape of a respective signal.

51. The ophthalmological laser therapy method according to claim 51, wherein the calibration method further comprises, inserting a material in the detection system, modifying the material in a focus area of the therapy laser beam by an energy/material interaction process, and detecting this modification with the electromagnetic or mechanical examination waves of the examination system, with the electromagnetic waves of the camera system having a third frequency from the visible spectrum or both of the foregoing.

55. The ophthalmological laser therapy method according to claim 54, wherein the calibration method further comprises, detecting the modification in a visual recording.

56. The ophthalmological laser therapy method according to claim 54, wherein the calibration method further comprises, forming a predefined pattern in the material by the energy/material interaction process.
